(19) United States
(12) Patent Application Publication
(10) Pub. No.: US 2024/0312041 A1
(43) Pub. Date: Sep. 19, 2024

(54) Monocular Camera-Assisted Technique with Glasses Accommodation for Precise Facial Feature Measurements at Varying Distances

(71) Applicant: Veero Analytics, LLC, Cambridge, MA (US)

(72) Inventors: Stan German, Boston, MA (US); Min T. Kim, Lexington, MA (US)

(73) Assignee: Veero Analytics, LLC, Cambridge, MA (US)

(21) Appl. No.: 18/607,841

(22) Filed: Mar. 18, 2024

Related U.S. Application Data
(60) Provisional application No. 63/490,610, filed on Mar. 16, 2023.

(51) Int. Cl. G06T 7/62; G06T 7/73; G06V 10/24; G06V 10/25; G06V 10/44; G06V 40/16; G06V 40/18

(52) U.S. Cl. CPC G06T 7/62; G06T 7/73; G06V 10/245; G06V 10/25; G06V 10/454; G06V 40/171; G06V 40/18; G06T 2207/30201

(57) ABSTRACT

Methods, techniques, and systems are provided that measure a person's facial features and pupillary distance using a single camera and provide accurate sizing and fitting of eyewear. Three primary steps include 3D face alignment, reference object (e.g., card) placement, and facial measurement calculation. A system for measuring point distances of facial feature includes a camera configured to produce output signals on a channel corresponding to one or more images; a memory including computer-executable instructions; and a processor coupled to the memory and operative to execute the computer-executable instructions for measuring the person's facial features.

### RELATED APPLICATION

This application claims priority to and benefit of U.S. Provisional Patent Application No. 63/490,610, filed Mar. 16, 2023, and entitled “Monocular Camera-Assisted Technique with Glasses Accommodation for Precise Facial Feature Measurements at Varying Distances,” the entire content of which is incorporated herein by reference.

### BACKGROUND

Prescribing a pair of corrective lenses typically requires knowledge of the wearer's pupillary distance (PD), the distance between the center of each pupil and the bridge of the wearer's nose. This measurement is a major bottleneck for online retailers who sell direct to consumers, especially for consumers who need strong prescriptions that require clinically accurate PD measurements. The eyewear industry has experienced significant growth in recent years, with a surge in online sales for prescription glasses, sunglasses, and sports eyewear. As more customers turn to online shopping for eyewear, it becomes increasingly important to ensure a proper fit, as ill-fitting eyewear can cause discomfort, reduced visual acuity, and even headaches. Traditional in-person methods for measuring facial features, such as using a ruler or a PD ruler, require physical presence and can be prone to human error. Consequently, there is a growing need for an accurate, user-friendly, and convenient solution to measure facial features and fit eyewear for online customers.

### SUMMARY

Aspects of the present disclosure include systems, methods, and software, e.g., including a software application/solution running on one or more processors, which enable measuring a person's facial features and pupillary distance (PD) to accurately size and fit them for eyewear. Embodiments of the present disclosure can include steps of 3D face alignment, reference card placement and detection, and facial measurement calculation.

One general aspect of the present disclosure includes a system for measuring facial features. The system can include: a camera configured to produce output signals on a channel corresponding to one or more images; a memory including computer-executable instructions; and a processor coupled to the memory and operative to execute the computer-executable instructions, the computer-executable instructions causing the processor to perform operations including: performing 3D face alignment of a user's face using a face landmark model (3D landmark detector) to identify positions of facial features or 3D landmark locations (locations of 3D landmarks), including 2D iris landmarks, where using the 3D landmark locations, a 3D pose of the user's face is estimated, where the 3D pose includes roll, pitch, yaw, x, y, and z coordinates; performing reference card placement and detection, including estimating the user's pupillary distance (PD) using an average camera field of view (FOV) and estimated iris diameter based on the 2D iris landmarks, where a derived rough pd scale is used to form a rectangular region-of-interest (ROI) on the user's forehead in an image captured by the camera, where the ROI illustrates correct placement of the reference card, and where the reference card is positioned in the ROI and detected by the camera; and performing one or more facial measurement calculations, including converting the size of the detected reference card in pixels to real-world dimensions, forming a pixel-to-distance ratio, and using the calculated pixel-to-distance ratio to convert distance between landmarks in pixels to actual facial measurements in metric units, where given an estimated camera FOV, a distance between the user and the camera is determined, and calculating one or more facial measurements, where the one or more facial measurements are scaled according to the user's distance from the camera.

Implementations may include one or more of the following features. The one or more calculated facial measurements (calculated by the system) can include a pupillary distance (PD). The one or more calculated facial measurements can include a face width (FW). The camera may include a color (a.k.a., red-green-blue, or “RGB”) camera including an RGB sensor (sensor or sensor array with R, G, B components, e.g., sub-arrays of photodetectors), where the RGB camera is configured to produce output signals on an RGB channel corresponding to one or more RGB images. The one or more images may include a plurality of frames of video from the RGB camera. The face landmark model may include a convolutional neural network. The face landmark model may include a deep landmark detection network. The reference object may include a reference card.

Another general aspect of the present disclosure includes a method of using a camera for measuring point distances of facial features. The method can include: performing 3D face alignment of a user's face using a face landmark model (3D landmark detector) to identify positions of facial features (3D landmarks), e.g., including 2D iris landmarks, where using the 3D landmark locations, a 3D pose of the user's face is estimated, where the 3D pose includes roll, pitch, yaw, x, y, and z coordinates; performing reference card placement and detection, including estimating the user's pupillary distance (PD) using an average camera field of view (FOV) and estimated iris diameter based on the 2D iris landmarks, where a derived rough pd scale is used to form a rectangular region-of-interest (ROI) on the user's forehead in an image captured by the camera, where the ROI illustrates correct placement of the reference card, and where the reference card is positioned in the ROI and detected by the camera; and performing one or more facial measurement calculations, including converting the size of the detected reference card in pixels to real-world dimensions, forming a pixel-to-distance ratio, and using the calculated pixel-to-distance ratio to convert distance between landmarks in pixels to actual facial measurements in metric units, where given an estimated camera FOV, a distance between the user and the camera is determined, and calculating one or more facial measurements, where the one or more facial measurements are scaled according to the user's distance from the camera

Implementations may include one or more of the following features. The one or more calculated facial measurements (calculated by the method) may include a pupillary distance (PD). The one or more calculated facial measurements may include a face width (FW). The camera may include an RGB (color) camera including an RGB sensor, where the RGB camera is configured to produce output signals on an RGB channel corresponding to one or more RGB images. The one or more images may include a plurality of frames of video from the RGB camera. The face (facial) landmark model may include a convolutional neural network. The face landmark model may include a deep landmark detection network. The reference object may include a reference card. Detecting the reference object may include edge detection.

A further general aspect of the present disclosure includes a computer readable storage medium including computer executable instructions for measuring point distances of facial features using a camera. The computer readable instructions included in the storage medium can include: performing 3D face alignment of a user's face using a face landmark model to identify positions of facial features or 3D landmarks, e.g., including 2D iris landmarks, where using the 3D landmark locations, a 3D pose of the user's face is estimated, where the 3D pose includes roll, pitch, yaw, x, y, and z coordinates; performing reference card placement and detection, including estimating the user's pupillary distance (PD) using an average camera field of view (FOV) and estimated iris diameter based on the 2D iris landmarks, where a derived rough pd scale is used to form a rectangular region-of-interest (ROI) on the user's forehead in an image captured by the camera, where the ROI illustrates correct placement of the reference card, and where the reference card is positioned in the ROI and detected by the camera; and performing one or more facial measurement calculations, including converting the size of the detected reference card in pixels to real-world dimensions, forming a pixel-to-distance ratio, and using the calculated pixel-to-distance ratio to convert distance between landmarks in pixels to actual facial measurements in metric units, where given an estimated camera FOV, a distance between the user and the camera is determined, and calculating one or more facial measurements, where the one or more facial measurements are scaled according to the user's distance from the camera.

Implementations may include one or more of the following features. The one or more calculated facial measurements (calculated by performance of the computer readable instructions) may include a pupillary distance (PD). The one or more calculated facial measurements may include a face width (FW). The camera may include an RGB camera including an RGB sensor, where the RGB camera is configured to produce output signals on an RGB channel corresponding to one or more RGB images. The one or more images may include a plurality of frames of video from the RGB camera. The face landmark model may include a convolutional neural network. The face landmark model may include a deep landmark detection network. The reference object may include a reference card. Detecting the reference object may include edge detection.

Embodiments and examples of the present disclosure may include corresponding computer systems, apparatus, and computer programs recorded on or resident in one or more computer storage (memory) devices or units (e.g., chips or circuits including RAM, ROM, etc.), each configured to perform the actions of the methods as described herein. A computer system of one or more computers can be configured to perform particular operations or actions, as described herein, by virtue of having software, firmware, hardware, or a combination of them installed on the system that in operation causes or cause the system to perform the operations or actions. One or more computer (software) programs can be configured to perform particular operations or actions by virtue of including instructions that, when executed by data processing apparatus, cause the apparatus to perform the operations or actions.

The features and advantages described herein are not all-inclusive; many additional features and advantages will be apparent to one of ordinary skill in the art in view of the drawings, specification, and claims. Moreover, it should be noted that the language used in the specification has been selected principally for readability and instructional purposes, and not to limit in any way the scope of the present disclosure, which is susceptible of many embodiments. What follows is illustrative, but not exhaustive, of the scope of the present disclosure.

### BRIEF DESCRIPTION OF THE DRAWINGS

The manner and process of making and using the disclosed embodiments may be appreciated by reference to the figures of the accompanying drawings. It should be appreciated that the components and structures illustrated in the figures are not necessarily to scale, emphasis instead being placed upon illustrating the principals of the concepts described herein. Furthermore, embodiments are illustrated by way of example and not limitation in the figures, in which:

FIG. 1A is a diagram showing an example flow chart for measuring point distances using a single camera, in accordance with the present disclosure;

FIG. 1B shows a flow chart for an example reference card detection process, in accordance with the present disclosure;

FIG. 2 is a diagram showing an example utilizing a reference card for PD measurement, in accordance with the present disclosure;

FIG. 3 is a block diagram for an example method for measuring point distances using a color camera, in accordance with the present disclosure; and

FIG. 4 is a block diagram of an example computer system operative to perform processing, in accordance with the present disclosure.

### DETAILED DESCRIPTION

The features and advantages described herein are not all-inclusive; many additional features and advantages will be apparent to one of ordinary skill in the art in view of the drawings, specification, and claims. Moreover, it should be noted that the language used in the specification has been selected principally for readability and instructional purposes, and not to limit in any way the scope of the inventive subject matter. The subject technology is susceptible of many embodiments. What follows is illustrative, but not exhaustive, of the scope of the subject technology.

Aspects of the present disclosure include systems, methods, including a software-based method (e.g., one or more software applications running on a suitable processor or processors) for accurately measuring distances between two or more 3D points using a single camera, e.g., a color camera included with a personal computer (PC) or wireless device such as a smart phone or tablet. Embodiments of the present disclosure can represent or provide for accurately measuring facial features at varying distances, utilizing a single camera as a primary imaging device.

Techniques (e.g., systems and/or methods) in accordance with the present disclosure can utilize a reference object, including, but not limited to, a card, with known dimensions, which the user places on their forehead or adjacent to their face. Techniques in accordance with the present disclosure can detect the reference object and facial landmarks using sophisticated computer vision algorithms and image processing techniques. By analyzing the reference object's size and position within the image relative to the facial landmarks, the techniques in accordance with present disclosure can calculate a scaling factor required to convert pixel distances into real-world measurements. Embodiments of the present disclosure can accordingly overcome limitations of traditional techniques that require known or fixed distances for accurate measurements, including measurements of pupillary distance (PD) for eyeglasses fitting.

Embodiments of the present disclosure can account for factors such as perspective distortion, focal convergence, and camera distance variations, ensuring precise facial feature measurements regardless of the user's distance from the camera. The techniques in accordance with the present disclosure can provide versatile and accurate solutions for measuring facial features in diverse scenarios and are accordingly particularly well suited for applications in online eyewear fitting, facial recognition, and personalized product design. Techniques for measuring a person's facial features and pupillary distance according to the present disclosure can provide for accurately sizing and fitting them for eyewear.

Exemplary embodiments of the present disclosure can include three general or primary steps or stages: (I) 3D face alignment, (II) reference object (e.g., credit card or other reference card) placement and detection, and (III) facial measurement calculation, as described in further detail below.

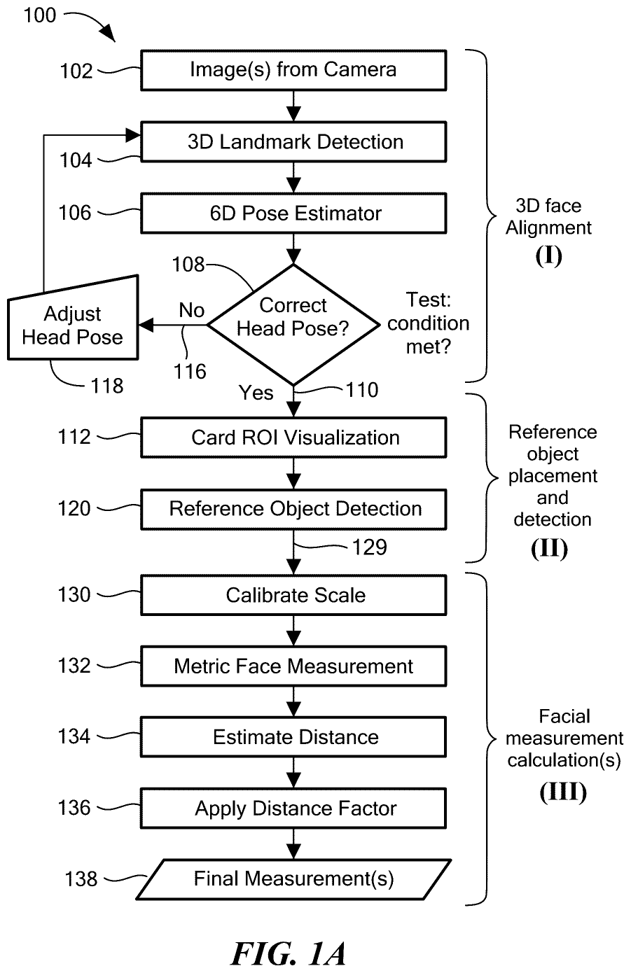

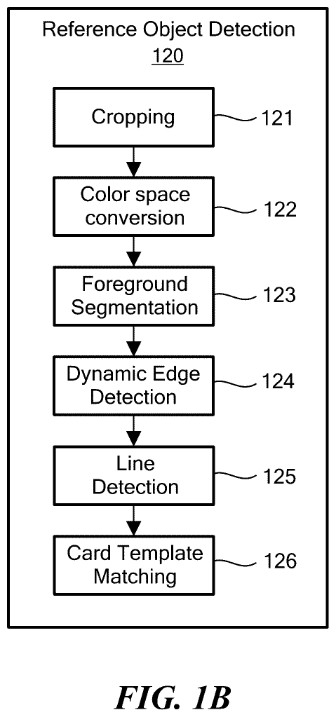

FIG. 1A is a diagram showing an example method (algorithm flow) 100 for measuring distances using a single camera in accordance with the present disclosure. Method 100 can provide robust measurement of distances between 3D points, e.g., including but not limited to, points defining the PD of a wearer of corrective lenses. Method 100 includes use of a camera 102, such as a color camera available from or included with a smartphone, e.g., an Android phone, or a personal computer (PC), which is used to provide (take) one or more images or video frames, e.g., including the face of a person for which a PD measurement is desired.

Method 100 includes a general step of 3D facial alignment (I). For 3D face alignment, the one or more images or video frames can be provided to or processed with a three-dimensional (3D) landmark model, detector or module (e.g., a software module) 104. This step 104 can employ a 3D face landmark model to identify the positions of essential facial features, including the eyes, pupils, nose, and mouth. The 3D landmark detector/module 104 can be used to identify and track points across multiple images and/or frames of video captured by camera 102. For the purpose of facial measurement, for example, 3D landmark detector 104 can be used to identify symmetric facial features that are mostly coplanar, such as left and right eye contours. In some embodiments, camera 102 can include a color camera such as one included with a wireless device (e.g., smart phone or tablet) or PC.

Using the 3D landmark locations (identified by landmark detector 104), the 3D pose of the face is estimated using a 6D pose estimator 106 (e.g., a software module). The 3D pose also includes roll, pitch, yaw, in addition to x, y, and z coordinates, for a total of six coordinates (6D). The user is then guided to align his/her/their face in this 6D pose through real-time feedback.

As shown at 108, method 100 can test whether the user (subject person in the one or more images) has her/his head in the correct position (pose/orientation). For example, one or more visual guides may be presented on viewing screen of the camera 102 to indicate correct positioning (position or placement) and/or pose of the person's head. In the event the person's head in not in the correct position, shown at 116, the method 100 can instruct the person (user) to position the camera sensor, so that it is parallel (or essentially parallel) to the plane of measurement, for some embodiments; or, the person shown in the image, can be instructed to adjust their head pose, as shown at 118.

For the condition where the head pose is correct, shown at 110, a region-of-interest (ROI) can be visualized, e.g., on or near the user's head, as shown at 112, for detection of a reference object such as a reference card. The reference object, e.g., reference card, can be detected within the ROI, as shown at 120. In some embodiments, the reference object may be a credit card or other similarly sized card.

Method 100 also includes a general step of reference object placement and detection (II). This step (II) involves roughly estimating the user's pupillary distance (PD) by considering the average camera field of view (FOV) for desktop and mobile (a.k.a., “selfie”) cameras and the user's estimated iris diameter based on 2D iris landmarks. A rough PD scale is derived and used to draw a rectangular region-of-interest (ROI) on the user's forehead (at 112), which illustrates (e.g., by showing on the camera or PC screen) the correct placement of a reference card (credit card-sized) on the user's forehead. After placement in the ROI, the reference object (e.g., card) can then be detected, as shown at 120. The detection 120 includes determining the size of the object/card (e.g., length, width, and/or area) in pixels.

In some embodiments, the reference object/card detection within the ROI (at 120) can utilize image processing techniques including converting to different color spaces, cropping the ROI, segmenting the foreground to exclude skin, hair, and other elements, morphologically denoising the segmented ROI, estimating edge detection parameters based on the color distributions of the ROI, and generating edge images. Straight lines can be identified in the computed edge images, clustered, and filtered to correspond with the expected card dimensions. When all four sides are detected and pass ratio validation, the card and its boundaries are established. FIG. 1B shows another embodiment of reference object detection in the ROI.

Method 100 further includes a general step of facial measurement calculation(s) (III). General step (III) includes calibrating scale (step 130), which involves converting the size of the detected reference card in pixels to its real-world dimensions. The calculated pixel-to-distance ratio (e.g., pixel-to-meter ratio) can be used to convert the distance between landmarks in pixels to actual facial measurements (at step 132) in real world distances (e.g., metric units), such as pupillary distance (PD) and face width (FW). Given an estimated camera FOV, the distance between the user and the camera is determined (calculated), at step 134; the estimated distance to the user can be particularly useful for calculating pupillary distance (PD), since it depends on the user's focus distance. For the estimated camera FOV, an average, known, guessed, or estimated FOV may be used, e.g., by considering the average camera FOV for desktop (PC) and/or mobile selfie cameras. Facial measurements, especially PD, are scaled according to the user's distance, as shown at 136. This results in highly accurate “final” measurements of the user's facial features, e.g., PD and FW, as shown at 138. The final measurements can be used to ensure precise sizing and fitting of eyewear for the user.

Embodiments of the present disclosure can accommodate glasses-on scanning, offering a unique advantage by allowing users to wear glasses during an initial scanning process. This feature can be used to cater to user comfort and ensures accurate facial measurements, particularly for pupillary distance and features near the glasses, which can be distorted by high index or strong prescription lenses or occluded by frames and lenses.

For users wearing glasses, a two-stage scanning process can be used to achieve accurate and reliable results, in accordance with embodiments of the present disclosure. The two stages can each include a process as shown and described for FIG. 1A (and/or FIG. 1B). During an initial scan, users wear their glasses and place the reference card on their forehead or near their face. The system (e.g., described for FIG. 1A) captures essential scale calibrations using pre-selected facial features that remain robust even when glasses are worn.

After removing the reference card, users then take a brief follow-up scan without their glasses. The system can then apply the scaling factor, obtained from the reference object during the first scan, to the measurements taken in the second scan without glasses. This can be done using the previously selected facial features, ensuring accuracy and reliability in the final measurements.

FIG. 1B shows another embodiment of reference object detection 120 in accordance with the present disclosure. The detection of a reference object can occur when the object (card) is in an ROI, which is illustrated/shown (pictorially represented) on or near the user's head in the image taken with a camera of the user's head. The ROI can be cropped, as shown at 121. A color space conversion can be performed, as shown at 122. A segmentation process can be performed, as shown at 123. The segmentation 123 can include foreground segmentation. A dynamic edge step may be performed, as shown at 124. A line detection step may be performed, as shown at 125. A reference object (e.g., card) template matching step may be performed, as shown at 126.

In some embodiments, a reference card may be segmented (at step 123), e.g., using a deep learning (DL) process. For example, in some embodiments, a machine learning (ML) model can be trained using a comprehensive dataset including images that present (showcase) a variety of reference cards affixed to the foreheads of various subjects under diverse lighting and/or environmental conditions. This training can enable the model to discern and learn the unique attributes of the reference cards. Post-training, the model can exhibit the capability to precisely isolate and segment the reference card in novel, previously unseen images by pinpointing its contours in relation to the forehead and adjacent regions. For this purpose, a convolutional neural network (CNN) or an equivalently suitable machine learning framework can be chosen for its adeptness in learning from the dataset.

The core of the training regimen can consist of iteratively refining the model's parameters to reduce the discrepancies between its segmentation predictions and the true outlines of the reference cards. When a new image is introduced via an image capture apparatus (e.g., camera in a smart device or PC as described at 102), the processing unit leverages the trained model to execute the segmentation task. This process entails the classification of each pixel within the image, determining whether it belongs to the reference card or not, thereby accurately demarcating the periphery of the card.

In some embodiments, segmentation techniques, such as simple linear iterative clustering (SLIC) and graph cut, can be used to improve the reference card detection process and enhance the overall accuracy of the system/process. These techniques may be used to improve the reference card detection process by more accurately distinguishing the card from the background and better handling variations in lighting, card orientation, and image noise.

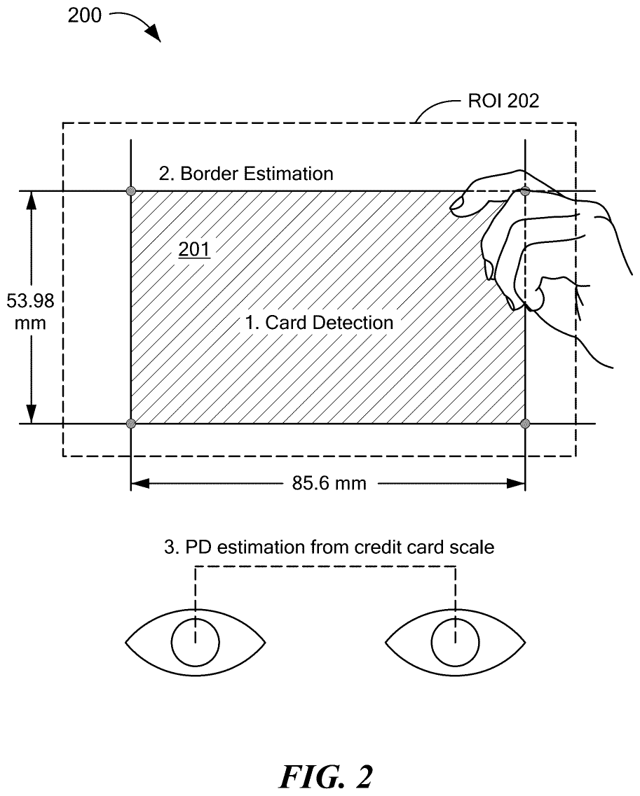

FIG. 2 is a diagram showing an example 200 utilizing a reference card for PD measurement in accordance with the present disclosure. As shown, a reference card 201 may be placed by a user in a position adjacent or over a portion of a user's head and within a region of interest (ROI) 202 visible in an image taken by a camera, for use in facial measurements in accordance with the present disclosure. Example dimensions are shown for reference card 201. A reference object may have other dimensions, sizes, and/or shapes within the scope of the present disclosure.

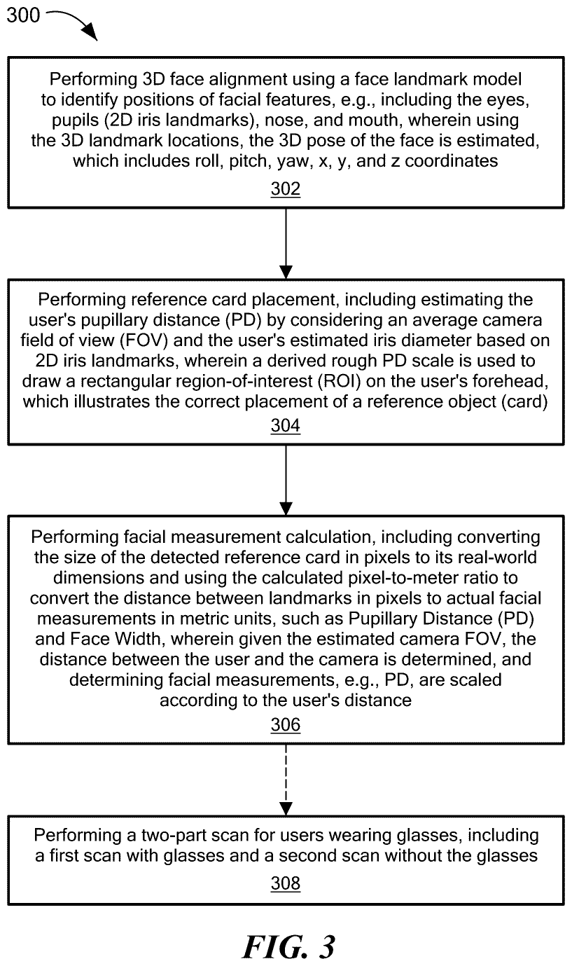

FIG. 3 is a block diagram for an example method 300 for measuring point distances using a color camera in accordance with the present disclosure. Method 300 can include performing 3D face alignment using a face landmark model to identify positions of essential facial features, including the eyes, pupils (2D iris landmarks), nose, and mouth, where using the 3D landmark locations, the 3D pose of the face is estimated, as described at 302. The 3D pose can include roll, pitch, yaw, x, y, and z coordinates in some embodiments, e.g., as described for FIG. 1A and FIG. 3; another suitable coordinate system (e.g., cylindrical, etc.) may be used for defining a 3D pose in other embodiments. As shown, method 300 also includes placing and detecting a reference object (card) in an ROI, as described at 304; this can include estimating the user's pupillary distance (PD) by considering an average camera field of view (FOV) and the user's estimated iris diameter based on 2D iris landmarks, with a derived rough PD scale being used to draw a rectangular region-of-interest (ROI) on or near the user's head, e.g., on the user's forehead. The ROI can be used to illustrate the correct placement of a reference object (card), guiding the user to place the reference object in the ROI for detection by the camera.

Method 300 further include performing (final) facial measurement calculation(s), as described at 306; the calculation(s) include converting the size of the detected reference card in pixels to its real-world dimensions and using the calculated pixel-to-meter ratio to convert the distance between landmarks in pixels to actual facial measurements in metric units, such as pupillary distance (PD) and face width (FW), wherein given the estimated camera FOV, the distance between the user and the camera is determined, and facial measurements are determined, e.g., PD, by scaling according to the distance of the user from the camera (where the user, in this context, refers to the person whose facial features are being measured), as described at 306.

For a case where a user is wearing glasses (optional), method 300 can include performing a two-part scan for user's wearing glasses, including a first scan with glasses and a second scan without the glasses, as described at 308. As noted above, during the first (initial) scan, users wear their glasses and place the reference card on their forehead or near their face. The system (e.g., described for FIG. 1A) captures essential scale calibrations using pre-selected facial features that remain robust even when glasses are worn. After removing the reference card, users then take a brief follow-up scan without their glasses. The system can then apply the scaling factor, obtained from the reference object during the first scan, to the measurements taken in the second scan without glasses. This can be done using the previously selected facial features, ensuring accuracy and reliability in the final measurements.

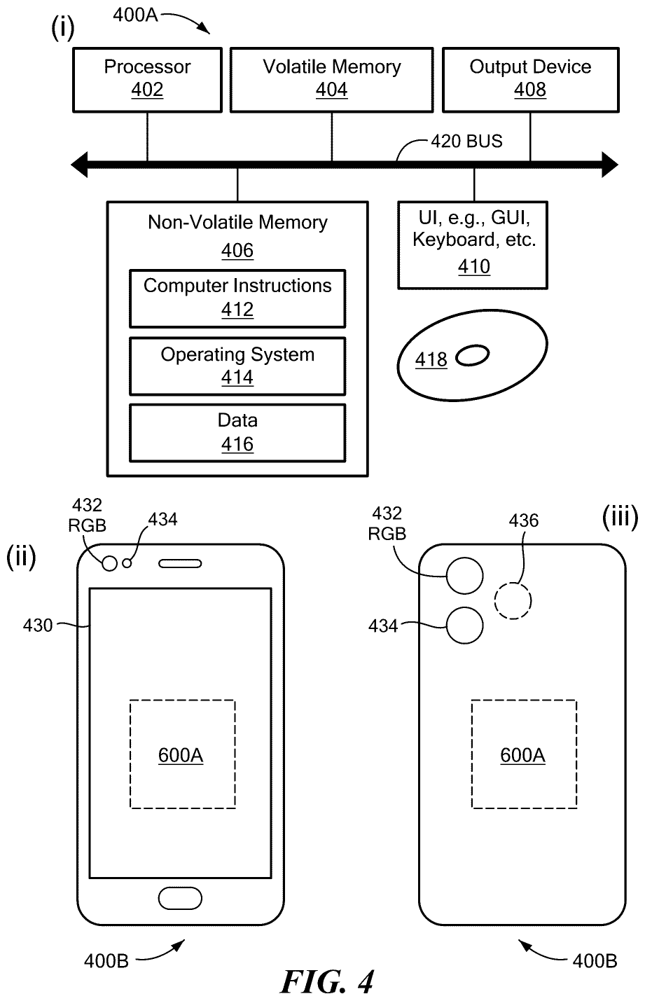

FIG. 4 is a block diagram, with views (i)-(iii), of example computer/computing systems 400 (400A-400B) operative (configured) to perform processing in accordance with the present disclosure, e.g., as described above for FIGS. 1A-3. Computer system 400 can perform all or at least a portion of the processing, e.g., steps in the algorithms, calculations, formulae, and methods described herein. View (i) shows system components of system 400A. Views (ii)-(iii) show example embodiments with system 400A implemented wireless cellular device 400B having a color (RGB) camera, e.g., a smartphone.

The computer system 400 (400A, 400B) includes one or more processors 402, one or more volatile memories 404, one or more non-volatile memories 4r06 (e.g., hard disk or cache), one or more output devices/components 408, and one or more user input or interfaces (UI) 410, e.g., a graphical user interface (GUI), a mouse, a keyboard, a display, and/or a touchscreen, etc. The non-volatile memory (e.g., cache or other non-transitory storage medium) 406 stores computer instructions 412 (a.k.a., machine-readable instructions or computer-readable instructions) such as software (computer program product), and/or an operating system 414 and data 416. In some examples/embodiments, the computer instructions 412 can be executed by the processor(s) 402 out of volatile memory 404. In some examples/embodiments, an article 418 (e.g., a storage device or medium such as a hard disk, an optical disc, magnetic storage tape, optical storage tape, flash drive, cache RAM, etc.) includes or stores the non-transitory computer-readable instructions. Bus 420 is also shown.

Processing may be implemented in hardware, software, or a combination of the two. Processing may be implemented in computer programs (e.g., software applications) executed on programmable computers/machines that each includes a processor, a storage medium or other article of manufacture that is readable by the processor (including volatile and non-volatile memory and/or storage elements), and optionally at least one input device, and one or more output devices. Program code may be applied to data entered using an input device or input connection (e.g., a port or bus) to perform processing and to generate output information.

The system 400 (400A, 400B) can perform processing, at least in part, via a computer program product or software application, (e.g., in a machine-readable storage device), for execution by, or to control the operation of, data processing apparatus (e.g., one or more programmable processors and/or computers). Each such program may be implemented in a high-level procedural or object-oriented programming language to communicate with a computer system, e.g., C, C#, C++, Python, etc.; any suitable programming language may be used. The program(s) may be implemented in assembly or machine language. The language may be a compiled or an interpreted language and it may be deployed in any form, including as a stand-alone program or as a module, component, subroutine, or other unit suitable for use in a computing environment.

A computer program may be deployed to be executed on one computer or on multiple computers at one site or distributed across multiple sites and interconnected by a communication network. A computer program may be stored on a storage medium or device (e.g., CD-ROM, hard disk, or magnetic diskette) that is readable by a general or special purpose programmable computer for configuring and operating the computer when the storage medium or device is read by the computer. Processing may also be implemented as a machine-readable storage medium, configured with a computer program, where upon execution, instructions in the computer program cause the computer to operate. Further, the terms “computer” or “computer system” may include reference to plural like terms, unless expressly stated otherwise.

As shown in views (ii)-(iii), in some embodiments a cellular device or smartphone 400B can include system 400A (or can provide real time or near real time wireless access to functionality of system 400A). Cellular device/smartphone 400B can include a user interface (e.g., a screen or touchscreen) 430 and a color (e.g., RGB or RGB-D) camera, as shown by separate RGB camera 432 and depth (D) 434 camera. Smartphone 400B may have more than two cameras, e.g., it can have three (432, 434, 436) as shown, though only a single camera is utilized for some embodiments of the present disclosure. The additional camera(s), e.g., camera 436, may be of any type, e.g., long-distance, high-resolution, black-and-white, another visible camera, or another LIDAR sensor. Cellular device/smartphone 400B can include functionality for wireless communication, e.g., according to any of a number of wireless transmission protocols or air interface standards. Cellular device/smartphone 400B can implement embodiments of the present disclosure, e.g., method 100 of FIG. 1, method 300 of FIG. 3, a method as claimed herein, and/or another method in accordance with the present disclosure.

### EXAMPLE EMBODIMENTS

An example embodiment can include a system for measuring coplanar point distances using a color camera, the system comprising:
a memory (e.g., one or more memory units or integrated circuits) including computer-executable instructions; and
one or more processors (e.g., a DSP and/or CPU) coupled to the memory and operative to execute the computer-executable instructions, the computer-executable instructions causing the processor(s) to perform:
(i) use a 3D landmark detector to identify facial features, e.g., including the eyes, pupils (2D iris landmarks), nose, and mouth in one or more images or video frames of a user's face captured by a camera (e.g., RGB camera of a smartphone) and estimating a 3D pose of the user's head/face, where the 3D pose includes 6D coordinates, which includes roll, pitch, yaw, x, y, and z coordinates;
(ii) (optional) as/if needed, guiding the user to align their his/her in this 6D pose through real-time feedback, e.g., visual on camera screen and/or audible prompts
(iii) perform reference object (card) ROI visualization, visually indicating the ROI (in the image of the user's face) on or near the user's face;
(iv) perform reference object detection;
(v) calibrate scale;
(vi) perform metric face measurement;
(vii) estimate distance;
(viii) apply a distance factor; and
(ix) perform final distance measurement calculation(s), e.g., for pupillary distance (PD) and/or face width (FW).

Another example embodiment can include a system for measuring coplanar point distances using a color camera, the system comprising:
a memory (e.g., one or more storage or memory units or integrated circuits including memory) including computer-executable instructions; and
one or more processors (e.g., a DSP and/or CPU) coupled to the memory and operative to execute the computer-executable instructions, the computer-executable instructions causing the processor(s) to perform, e.g., the processes and/or steps described herein for FIGS. 1A-3.

In some embodiments, a color camera can be provided by or included in a smart phone, e.g., one running an Android operating system, or a personal computer (PC). Some embodiments may utilize or be used/developed with Google's MediaPipe, which provides open-source, cross-platform, customizable machine learning (ML) models, including ML models built with the hardware limitations of mobile devices in mind.

Processing may be performed by one or more programmable processors executing one or more computer programs to perform the functions of the system. All or part of the system may be implemented as special purpose logic circuitry, e.g., an FPGA (field programmable gate array) and/or an ASIC (application-specific integrated circuit). In some examples, digital logic circuitry, e.g., one or more FPGAs, can be operative as a processor as described herein.

Accordingly, embodiments of the inventive subject matter can afford various benefits relative to prior art techniques. For example, embodiments and examples of the present disclosure can provide a convenient and accurate method for measuring facial features, such as pupillary distance and face width, without the need for in-person visits or professional assistance. Embodiments can improve the customer experience by ensuring a perfect fit for eyewear purchased online, increasing customer satisfaction and loyalty. Additionally, embodiments can reduce the number of returns and exchanges due to sizing errors, lowering costs for both customers and retailers. Embodiments can increase conversion rates for online eyewear retailers by boosting customer confidence in purchasing eyewear online. Embodiments can help eyewear retailers reach a broader customer base, including those in remote locations or those who cannot visit a physical store. Embodiments of the present disclosure can provide improved camera distance estimation by considering camera distance and its effects on focal convergence and perspective distortion, ensuring accurate facial measurements by compensating for these factors. Embodiments of the present disclosure can provide a user-friendly interface, e.g., by providing real-time feedback and guidance to the user, ensuring proper face alignment and reference card placement, making it accessible and straightforward for users with varying levels of technical expertise.

Moreover, embodiments of the present disclosure can offer improved customer experience: by providing accurate facial measurements and ensuring a perfect fit, embodiments of the present disclosure can enhance the customer experience, increasing customer satisfaction and loyalty. Embodiments of the present disclosure can offer reduced returns and exchanges: by minimizing sizing errors, embodiments of the present disclosure can help decrease the number of returns and exchanges, reducing costs for both customers and retailers. Embodiments of the present disclosure can offer increased sales conversion rates: embodiments of the present disclosure can boost customer confidence in purchasing eyewear online, leading to higher conversion rates for online eyewear retailers. Embodiments of the present disclosure can offer expanded market reach: embodiments of the present disclosure can enable eyewear retailers to reach customers in remote locations or those who are unable to visit a physical store, increasing the potential customer base. Embodiments of the present disclosure can offer competitive advantage: embodiments of the present disclosure can provide eyewear retailers with the ability to differentiate themselves from competitors and position themselves as industry leaders in customer service and innovation.

Additionally, embodiments of the present disclosure can mitigate or guard against inaccurate measurements by utilizing advanced computer vision and image processing techniques, accounting for factors such as perspective distortion and focal convergence, to ensure accurate facial measurements. Embodiments of the present disclosure reduce inconvenience to the user (eyeglasses purchaser) by providing a user-friendly interface with real-time feedback and guidance, allowing users to obtain accurate measurements without professional assistance or in-person visits. Embodiments of the present disclosure can mitigate or reduce high return and exchange rates by offering precise measurements and ensuring a perfect fit, the solution helps minimize returns and exchanges due to sizing errors, reducing costs for customers and retailers. Embodiments of the present disclosure can also provide improved access to customers (users) by enabling eyewear retailers to cater to customers in remote locations or those who cannot visit a physical store, expanding their potential customer base.

It will be understood that, while various embodiments of the concepts, systems, devices, structures, and techniques sought to be protected are described above with reference to the related drawings, alternative embodiments can be devised without departing from the scope of the concepts, systems, devices, structures, and techniques described.

It is noted that various connections and positional relationships (e.g., over, below, adjacent, etc.) may be used to describe elements and components in the description and drawings. These connections and/or positional relationships, unless specified otherwise, can be direct or indirect, and the described concepts, systems, devices, structures, and techniques are not intended to be limiting in this respect. Accordingly, a coupling of entities can refer to either a direct or an indirect coupling, and a positional relationship between entities can be a direct or indirect positional relationship.

As an example of an indirect positional relationship, positioning element “A” over element “B” can include situations in which one or more intermediate elements (e.g., element “C”) is between elements “A” and elements “B” as long as the relevant characteristics and functionalities of elements “A” and “B” are not substantially changed by the intermediate element(s).

Also, the following definitions and abbreviations are to be used for the interpretation of the claims and the specification. The terms “comprise,” “comprises,” “comprising,” “include,” “includes,” “including,” “has,” “having,” “contains” or “containing,” or any other variation are intended to cover a non-exclusive inclusion. For example, an apparatus, a method, a composition, a mixture, or an article, including a list of elements is not necessarily limited to only those elements but can include other elements not expressly listed or inherent to such apparatus, method, composition, mixture, or article.

Additionally, the term “exemplary” means “serving as an example, instance, or illustration. Any embodiment or design described as “exemplary” is not necessarily to be construed as preferred or advantageous over other embodiments or designs. The terms “one or more” and “at least one” indicate any integer number greater than or equal to one, i.e., one, two, three, four, etc.; though those terms may include reference to fractional values where context admits. The term “plurality” indicates any integer number greater than one; though that term may include reference to a fractional value where context admits. The term “connection” can include an indirect “connection” and a direct “connection”.

References in the specification to “embodiments,” “one embodiment, “an embodiment,” “an example embodiment,” “an example,” “an instance,” “an aspect,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it may affect such feature, structure, or characteristic in other embodiments whether explicitly described or not.

Relative or positional terms including, but not limited to, the terms “upper,” “lower,” “right,” “left,” “vertical,” “horizontal, “top,” “bottom,” and derivatives of those terms relate to the described structures and methods as oriented in the drawing figures. The terms “overlying,” “atop,” “on top, “positioned on” or “positioned atop” mean that a first element, such as a first structure, is present on a second element, such as a second structure, where intervening elements such as an interface structure can be present between the first element and the second element. The term “direct contact” means that a first element, such as a first structure, and a second element, such as a second structure, are connected without any intermediary elements.

Use of ordinal terms such as “first,” “second,” “third,” etc., in the claims to modify a claim element does not by itself connote any priority, precedence, or order of one claim element over another, or a temporal order in which acts of a method are performed but are used merely as labels to distinguish one claim element having a certain name from another element having a same name (but for use of the ordinal term) to distinguish the claim elements.

The terms “approximately” and “about” may be used to mean within ±20% of a target (or nominal) value in some embodiments, within plus or minus (±) 10% of a target value in some embodiments, within +5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

The disclosed subject matter is not limited in its application to the details of construction and to the arrangements of the components set forth in the following description or illustrated in the drawings. The disclosed subject matter is capable of other embodiments and of being practiced and carried out in various ways.

Also, the phraseology and terminology used in this patent are for the purpose of description and should not be regarded as limiting. As such, the conception upon which this disclosure is based may readily be utilized as a basis for the designing of other structures, methods, and systems for carrying out the several purposes of the disclosed subject matter. Therefore, the claims should be regarded as including such equivalent constructions as far as they do not depart from the spirit and scope of the disclosed subject matter.

Although the disclosed subject matter has been described and illustrated in the foregoing exemplary embodiments, the present disclosure has been made only by way of example. Thus, numerous changes in the details of implementation of the disclosed subject matter may be made without departing from the spirit and scope of the disclosed subject matter.

Accordingly, the scope of this patent should not be limited to the described implementations but rather should be limited only by the spirit and scope of the following claims.

What is claimed is:

1. A system for measuring facial features, the system comprising:
a camera configured to produce output signals on a channel corresponding to one or more images;
a memory including computer-executable instructions; and
a processor coupled to the memory and operative to execute the computer-executable instructions, the computer-executable instructions causing the processor to perform operations including:
(i) performing 3D face alignment of a user's face using a face landmark model to identify positions of facial features, including 2D iris landmarks, wherein using the 3D landmark locations, a 3D pose of the user's face is estimated, wherein the 3D pose includes roll, pitch, yaw, x, y, and z coordinates;
(ii) performing reference card placement and detection, including estimating the user's pupillary distance (PD) using an average camera field of view (FOV) and estimated iris diameter based on the 2D iris landmarks, wherein a derived rough PD scale is used to form a rectangular region-of-interest (ROI) on the user's forehead in an image captured by the camera, wherein the ROI illustrates correct placement of the reference card, and wherein the reference card is positioned in the ROI and detected by the camera; and
(iii) performing one or more facial measurement calculations, including converting the size of the detected reference card in pixels to real-world dimensions, forming a pixel-to-distance ratio, and using the calculated pixel-to-distance ratio to convert distance between landmarks in pixels to actual facial measurements in metric units, wherein given an estimated camera FOV, a distance between the user and the camera is determined, and calculating one or more facial measurements, wherein the one or more facial measurements are scaled according to the user's distance from the camera.

2. The system of claim 1, wherein the one or more calculated facial measurements include a pupillary distance (PD).

3. The system of claim 1, wherein the one or more calculated facial measurements include a face width (FW).

4. The system of claim 1, wherein the camera comprises an RGB camera including an RGB sensor, wherein the RGB camera is configured to produce output signals on an RGB channel corresponding to one or more RGB images.

5. The system of claim 1, wherein the one or more images comprise a plurality of frames of video from the RGB camera.

6. The system of claim 1, wherein the face landmark model comprises a convolutional neural network.

7. The system of claim 1, wherein the face landmark model comprises a deep landmark detection network.

8. The system of claim 1, wherein the reference object comprises a reference card.

9. A method of using a camera for measuring point distances of facial features, the method comprising:
(i) performing 3D face alignment of a user's face using a face landmark model to identify positions of facial features, including 2D iris landmarks, wherein using the 3D landmark locations, a 3D pose of the user's face is estimated, wherein the 3D pose includes roll, pitch, yaw, x, y, and z coordinates;
(ii) performing reference card placement and detection, including estimating the user's pupillary distance (PD) using an average camera field of view (FOV) and estimated iris diameter based on the 2D iris landmarks, wherein a derived rough PD scale is used to form a rectangular region-of-interest (ROI) on the user's forehead in an image captured by the camera, wherein the ROI illustrates correct placement of the reference card, and wherein the reference card is positioned in the ROI and detected by the camera; and
(iii) performing one or more facial measurement calculations, including converting the size of the detected reference card in pixels to real-world dimensions, forming a pixel-to-distance ratio, and using the calculated pixel-to-distance ratio to convert distance between landmarks in pixels to actual facial measurements in metric units, wherein given an estimated camera FOV, a distance between the user and the camera is determined, and calculating one or more facial measurements, wherein the one or more facial measurements are scaled according to the user's distance from the camera.

10. The method of claim 9, wherein the one or more calculated facial measurements include a pupillary distance (PD).

11. The method of claim 9, wherein the one or more calculated facial measurements include a face width (FW).

12. The method of claim 9, wherein the camera comprises an RGB camera including an RGB sensor, wherein the RGB camera is configured to produce output signals on an RGB channel corresponding to one or more RGB images.

13. The method of claim 9, wherein the one or more images comprise a plurality of frames of video from the RGB camera.

14. The method of claim 9, wherein the face landmark model comprises a convolutional neural network.

15. The method of claim 9, wherein the face landmark model comprises a deep landmark detection network.

16. The method of claim 9, wherein the reference object comprises a reference card.

17. The method of claim 9, wherein detecting the reference object comprises edge detection.

18. A computer readable storage medium including computer executable instructions for measuring point distances of facial features using a camera, which when read by a processor cause the processor to perform operations including:
(i) performing 3D face alignment of a user's face using a face landmark model to identify positions of facial features, including 2D iris landmarks, wherein using the 3D landmark locations, a 3D pose of the user's face is estimated, wherein the 3D pose includes roll, pitch, yaw, x, y, and z coordinates;
(ii) performing reference card placement and detection, including estimating the user's pupillary distance (PD) using an average camera field of view (FOV) and estimated iris diameter based on the 2D iris landmarks, wherein a derived rough PD scale is used to form a rectangular region-of-interest (ROI) on the user's forehead in an image captured by the camera, wherein the ROI illustrates correct placement of the reference card, and wherein the reference card is positioned in the ROI and detected by the camera; and
(iii) performing one or more facial measurement calculations, including converting the size of the detected reference card in pixels to real-world dimensions, forming a pixel-to-distance ratio, and using the calculated pixel-to-distance ratio to convert distance between landmarks in pixels to actual facial measurements in metric units, wherein given an estimated camera FOV, a distance between the user and the camera is determined, and calculating one or more facial measurements, wherein the one or more facial measurements are scaled according to the user's distance from the camera.

19. The storage medium of claim 18, wherein the one or more calculated facial measurements include a pupillary distance (PD).

20. The storage medium of claim 18, wherein the one or more calculated facial measurements include a face width (FW).

21. The storage medium of claim 18, wherein the camera comprises an RGB camera including an RGB sensor, wherein the RGB camera is configured to produce output signals on an RGB channel corresponding to one or more RGB images.

22. The storage medium of claim 18, wherein the one or more images comprise a plurality of frames of video from the RGB camera.

23. The storage medium of claim 18, wherein the face landmark model comprises a convolutional neural network.

24. The storage medium of claim 18, wherein the face landmark model comprises a deep landmark detection network.

25. The storage medium of claim 18, wherein the reference object comprises a reference card.

26. The storage medium of claim 18, wherein detecting the reference object comprises edge detection.
